SEQUENCE LISTING (38 sequences)

SEQ ID NO: 1 - DNA, 1239 bp, Homo sapiens
aaaatcaatataaagcagccatgtctggaggagaccaggaggtcaagaagccttagtttctcaagcccttagcaccaaattctctgagatcagctcttccttcagttacactgagcgtttcccctctgcagtgatggagaagggagaactcttattttttctcatgagcatctctggggctgttttccttagataaataagtggttctatttaatgtgaagcctgttttatgaacaggatgaatgtggtatatattcagaataactaakgtttggaagttgttttgttttgctaaaacaaagttttagcaaacgattttttttttcaaatttgtgtcttctgttctcaaagyatctctgatgtaagagataatgcgccacgatgggcatcagaagacctcagctcaaatcccagttctgccagctatgagctgtgtggcaccaacaggtgtcctgttctcccagggtctcccttttcccatttgaaatataaaaaataacaattcctgccttcacgtgtttttttagggggttaaatggtaaaggtgtttatatctgctaaggtaatttacttgatatatgtttggttattgaagatatatgagttatgttagctatttcatgtttaggctgctgtatttttagtaggctatattaaatagaggatttcattataaaggacaaagtctcctaatcttcgatataggattgacatactttttaaatatacaaggcatagaatatggccatttccgttaaatcataaattcccaactggttattaatctaagaattcagaattttaagtaattgtttttgcatcagattgtttacttcagtgctctcaattatgacggtgcattggaaccacttgggttaacatttttttgtttttattaccaatacctaggcttcaacctagtacaatgaaaccagaatgtacagagtgggcactgggacgaaggagaacaagaccaaaggacattttatttttatctctatcagtgggtcaaagtcctttcagaaggagcatatagtgggcctaggtgattggccacttyatccatcaaagaggcacacacacttaattagcatggagtgttataaaaagcttggagtgcaagctcacggttgtcttaacaagaggagaaggcttcaatggatccttttgtggtccttgtgctctgtctctcatgtttgcttctcctttcaatctggagacagagctctgggagaggaa

SEQ ID NO: 2 - DNA, 11 bp, Artificial Sequence
  Oligonucleotide of polymorphic site 269
actaatgtttg

SEQ ID NO: 3 - DNA, 11 bp, Artificial Sequence
  Oligonucleotide of polymorphic site 352
caaagcatctc

SEQ ID NO: 4 - DNA, 11 bp, Artificial Sequence
  Oligonucleotide of polymorphic site 1060
cactttatcca

SEQ ID NO: 5 - DNA, 11 bp, Artificial Sequence
  Oligonucleotide of polymorphic site 269
actaaggtttg

SEQ ID NO: 6 - DNA, 11 bp, Artificial Sequence
  Oligonucleotide of polymorphic site 352
caaagtatctc

SEQ ID NO: 7 - DNA, 11 bp, Artificial Sequence
  Oligonucleotide of polymorphic site 1060
cacttcatcca

SEQ ID NO: 8 - DNA, 22 bp, Artificial Sequence
  Primer of polymorphic site 269 & 352
caggaggtcaagaagccttagt

SEQ ID NO: 9 - DNA, 19 bp, Artificial Sequence
  Primer of polymorphic site 269 & 352
ccatcgtggcgcattatct

SEQ ID NO: 10 - DNA, 20 bp, Artificial Sequence
  Primer of polymorphic site 1060
acggtgcattggaaccactt

SEQ ID NO: 11 - DNA, 21 bp, Artificial Sequence
  Primer of polymorphic site 1060
cccagagctctgtctccagat

SEQ ID NO: 12 - DNA, 17 bp, Artificial Sequence
  Primer of polymorphic site 1060
agtgggcactgggacga

SEQ ID NO: 13 - DNA, 20 bp, Artificial Sequence
  Primer of polymorphic site 1060
gatccattgaagccttctcc

SEQ ID NO: 14 - DNA, 23 bp, Artificial Sequence
  Primer of polymorphic site 1060
gtaattgtttttgcatcagattg

SEQ ID NO: 15 - DNA, 23 bp, Artificial Sequence
  Primer of polymorphic site 1060
tccatgctaattaagtgtgtgtg

SEQ ID NO: 16 - DNA, 22 bp, Artificial Sequence
  Primer of polymorphic site 269 & 352
ctgagatcagctcttccttcag

SEQ ID NO: 17 - DNA, 24 bp, Artificial Sequence
  Primer of polymorphic site 269 & 352
aggcaggaattgttattttttata

SEQ ID NO: 18 - DNA, 20 bp, Artificial Sequence
  Primer of polymorphic site 352
tggggctgttttccttagat

SEQ ID NO: 19 - DNA, 22 bp, Artificial Sequence
  Primer of polymorphic site 352
atttaaccccctaaaaaaacac

SEQ ID NO: 20 - DNA, 11 bp, Artificial Sequence
  Oligonucleotide of polymorphic site 269
caaacattagt

SEQ ID NO: 21 - DNA, 11 bp, Artificial Sequence
  Oligonucleotide of polymorphic site 269
caaaccttagt

SEQ ID NO: 22 - DNA, 11 bp, Artificial Sequence
  Oligonucleotide of polymorphic site 352
gagatgctttg

SEQ ID NO: 23 - DNA, 11 bp, Artificial Sequence
  Oligonucleotide of polymorphic site 352
gagatactttg

SEQ ID NO: 24 - DNA, 11 bp, Artificial Sequence
  Oligonucleotide of polymorphic site 1060
tggataaagtg

SEQ ID NO: 25 - DNA, 11 bp, Artificial Sequence
  Oligonucleotide of polymorphic site 1060
tggatgaagtg

SEQ ID NO: 26 - DNA, 11 bp, Artificial Sequence
  Oligonucleotide of polymorphic site 269
tcagaataact

SEQ ID NO: 27 - DNA, 11 bp, Artificial Sequence
  Oligonucleotide of polymorphic site 352
tctgttctcaa

SEQ ID NO: 28 - DNA, 11 bp, Artificial Sequence
  Oligonucleotide of polymorphic site 1060
tgattggccac

SEQ ID NO: 29 - DNA, 11 bp, Artificial Sequence
  Oligonucleotide of polymorphic site 269
agttattctga

SEQ ID NO: 30 - DNA, 11 bp, Artificial Sequence
  Oligonucleotide of polymorphic site 352
ttgagaacaga

SEQ ID NO: 31 - DNA, 11 bp, Artificial Sequence
  Oligonucleotide of polymorphic site 1060
gtggccaatca

SEQ ID NO: 32 - DNA, 10 bp, Artificial Sequence
  Oligonucleotide of polymorphic site 269
acttccaaac

SEQ ID NO: 33 - DNA, 11 bp, Artificial Sequence
  Oligonucleotide of polymorphic site 352
acatcagagat

SEQ ID NO: 34 - DNA, 11 bp, Artificial Sequence
  Oligonucleotide of polymorphic site 1060
ctttgatggat

SEQ ID NO: 35 - DNA, 10 bp, Artificial Sequence
  Oligonucleotide of polymorphic site 269
gtttggaagt

SEQ ID NO: 36 - DNA, 11 bp, Artificial Sequence
  Oligonucleotide of polymorphic site 352
atctctgatgt

SEQ ID NO: 37 - DNA, 11 bp, Artificial Sequence
  Oligonucleotide of polymorphic site 1060
atccatcaaag

SEQ ID NO: 38 - DNA, 29 bp, Artificial Sequence
  29-mer nucleotide tail to PCR-primers
agtcacgacgttgtaaaacgacggccagt

(19) United States
(12) Patent Application Publication
(10) Pub. No.: US 2003/0059774 A1
(43) Pub. Date: Mar. 27, 2003

(54) Detection of CYP2C19 polymorphisms

(76) Inventors: Carl Risinger, Uppsala (SE); Maria Kristina Andersson, Uppsala (SE); Tommy Lewander, Uppsala (SE); Erik Olaisson, Uppsala (SE)

Correspondence Address: MORRISON & FOERSTER LLP, 3811 VALLEY CENTRE DRIVE, SUITE 500, SAN DIEGO, CA, 92130-2332, US

(21) Appl. No.: 09/943,531

(22) Filed: Aug. 30, 2001

(30) Foreign Application Priority Data
Aug. 30, 2000 (GB) 0021286.0

(51) Int. Cl. C12Q 1/68; C07H 21/04

(52) U.S. Cl. 435/6; 536/23.2; 536/24.3

(57) ABSTRACT

The invention provides methods, PCR primers, sequence determination oligonucleotides, isolated polynucleotides, and kits for determining a human's capacity to metabolize a substrate of the CYP2C19 enzyme using genetic analysis.

The present invention is directed to detection of certain polymorphisms in the 5′ regulatory region of the gene encoding cytochrome P450 2C19, also known as CYP2C19, S-mephenytoin-4′-hydroxylase, to predict variations in an individual's ability to metabolize certain drugs.

### BACKGROUND OF THE INVENTION

Xenobiotics are pharmacologically, endocrinologically, or toxicologically active substances foreign to a biological system. Most xenobiotics, including pharmaceutical agents, are metabolized through two successive reactions. Phase I reactions (functionalization reactions), include oxidation, reduction, and hydrolysis, in which a derivatizable group is added to the original molecule. Functionalization prepares the drug for further metabolism in phase II reactions. During phase II reactions (conjugative reactions, which include glucoronidation, sulfation, methylation and acetylation), the functionalized drug is conjugated with a hydrophilic group. The resulting hydrophilic compounds are inactive and excreted in bile or urine. Thus, metabolism can result in detoxification and excretion of the active substance. Alternatively, an inert xenobiotic may be metabolized to an active compound. For example, a pro-drug may be converted to a biologically active therapeutic or toxin.

The cytochrome P450 (CYP) enzymes are involved in the metabolism of many different xenobiotics. CYPs are a superfamily of heme-containing enzymes, found in eukaryotes (both plants and animals) and prokaryotes, and are responsible for Phase I reactions in the metabolic process. In total, over 500 genes belonging to the CYP superfamily have been described and divided into subfamilies, CYP1-CYP27. In humans, more than 35 genes and 7 pseudogenes have been identified. Members of three CYP gene families, CYP1, CYP2, and CYP3, are responsible for the majority of drug metabolism. The human CYPs which are of greatest clinical relevance for the metabolism of drugs and other xenobiotics are CYP1A2, CYP2A6, CYP2C9, CYP2C19, CYP2C19, CYP2E1 and CYP3A4. The liver is the major site of activity of these enzymes, however CYPs are also expressed in other tissues.

The CYP2C19 enzyme is responsible for metabolism of anticonvulsants such as mephobarbital and hexobarbital, proton pump inhibitors such as omeprazole and pentaprazole, antimalarial drugs such as proguanil and chlorproguanyl, antidepressants such as citalopram, and the benzodiazepines diazepam and desmethyldiazepam. In addition, CYP2C19 acts in sidechain oxidation of propranolol and in demethylation of imipramine.

CYP2C19 is a polymorphic enzyme, that is, more than one form of the enzyme is present within the human population. The different forms of the CYP2C19 enzyme have differing abilities to metabolize substrates, which impacts on the rate at which the substrates are removed from the body. The form of CYP2C19 that an individual inherits will determine how quickly a substrate is removed from the individual's body. Because CYP2C19 is polymorphic, individuals differ in their ability to metabolize the drugs that are substrates of CYP2C19, and consequently, wide variations in responses to such drugs, including susceptibility to side effects, have been observed.

On the basis of ability of metabolize a marker drug such as mephenytoin or omeprazol, individuals may be characterized as poor metabolizers (PM), intermediate metabolizers (IM), extensive metabolizers (EM) or ultra extensive metabolizers (UEM or UM) for CYP2C19 substrates. Poor metabolizers retain the CYP2C19 substrate in their bodies for a relatively long period of time, and are susceptible to toxicity and side effects at “normal” dosages. Ultraextensive metabolizers clear the CYP2C19 substrate from their bodies quickly, and require higher than “normal” dosages to achieve a therapeutic effect. Intermediate and extensive metabolizers retain the CYP2C19 substrate in their bodies for times between those of PMs and UEMs, and are more likely to respond to “normal” dosages of the drug. However, individuals characterized as IM or EM may differ in drug clearance by as much as 10-fold, and variations in toxicity, side effects, and efficacy for a particular drug may occur among these individuals.

The existence of more than one form of the CYP2C19 enzyme is caused by polymorphisms in the gene which encodes the CYP2C19 enzyme (the gene being denoted in italics, as CYP2C19, SEQ ID NO:1). In fact, more than 10 polymorphisms in the CYP2C19 gene have been described (see http://www.imm.ki.se/cypalleles/ for listing). The distribution of particular CYP2C19 polymorphisms differs widely among ethnic groups, with concomitant differences in CYP2C19 activity and responses to drugs which are CYP2C19 substrates. Approximately 2.5 to 6% of Caucasians are deficient in CYP2C19, while this deficiency is much more common in Japanese (18-23%) and Chinese (15-17%) individuals. The most common polymorphism responsible for the CYP2C19 PM phenotype is a single base pair substitution in exon 5 at position 681 of the coding sequence, designated CYP2C19*2 or CYP2C19m1, which results in a truncated, inactive protein. A second single base pair mutation in exon 4 at position 636 of the coding sequence, designated CYP2C19*3 or CYP2C19m2, also results in a truncated, inactive protein. The CYP2C19*2 and CYP2C19*3 mutations account for almost all PMs in Japanese and Chinese populations, while the CYP2C19*2 mutation causes about 87% of PMs in Caucasian populations. CYP2C19*1 encodes an active enzyme and is commonly known as the wild type gene.

U.S. Pat. No. 5,786,191 discloses methods of screening for drugs metabolized by CYP2C19 using the CYP2C19 polypeptide. U.S. Pat. No. 5,912,120 and related WO 95/30766 disclose methods of diagnosis of a deficiency in CYP2C19 activity caused by the CYP2C19*2 and CYP2C19*3 polymorphisms. WO 00/12757 discloses a primer extension assay and kit for detection of single nucleotide polymorphisms (SNPs) in cytochrome P450 isoforms, including the CYP2C19ml and CYP2C19m2 polymorphisms.

Although it is known that use of omeprazole as a marker drug reveals CYP2C19 UEMs, very little characterization of the genetics of these individuals exists. A need remains for diagnostic or prognostic methods and tools for use in predicting a CYP2C19 UEM individual's likely response to a drug which is a CYP2C19 substrate, and in selecting subjects for clinical trials of such drugs.

### SUMMARY OF THE INVENTION

The present inventors have discovered that individuals who are homozygous or heterozygous for certain haplotypes consisting of polymorphic sites in the 5′ flanking region of the CYP2C19 gene exhibit characteristic metabolic ratios for omeprazole. Using this information, the capacity of individuals to metabolize drugs which are substrates of the CYP2C19 enzyme may be predicted by genotyping those polymorphisms.

In one embodiment, the invention provides a method for determining a human's capacity to metabolize a substrate of a CYP2C19 enzyme, said method comprising the steps of: isolating single stranded nucleic acids from the human, said nucleic acids encoding 5′ flanking regions of CYP2C19 genes present on each homologous chromosome 10 of the human, wherein said region is represented by a sequence as set forth in SEQ ID NO:1; and detecting nucleotides present at polymorphic sites represented by positions 352 and 1060 of SEQ ID NO:1.

In another embodiment, the invention provides a sequence determination oligonucleotide suitable for detecting polymorphic sites in a 5′ flanking region of a CYP2C19 gene, said oligonucleotide comprising a sequence selected from the group consisting of an oligonucleotide complementary to the polymorphic region corresponding to position 269 of SEQ ID NO:1; an oligonucleotide complementary to the polymorphic region corresponding to position 352 of SEQ ID NO:1; and an oligonucleotide complementary to the polymorphic region corresponding to position 1060 of SEQ ID NO:1, both on the coding (sense) strand (SEQ ID NO:s 3-8, Table 6; SEQ ID NO:s 27-29, Table 8; and SEQ ID NO:s 36-38, Table 9) and on the non-coding (anti-sense) strand (SEQ ID NO:s 21-26, Table 7; SEQ ID NO:s 30-32, Table 8; and SEQ ID NO:s 33-35, Table 9).

In yet another embodiment, the invention provides an oligonucleotide primer pair suitable for amplifying a polymorphic region of a 5′ flanking region of a CYP2C19 gene, wherein the polymorphic region corresponds to position 269 of SEQ ID NO:1, position 352 of SEQ ID NO:1, or position 1060 of SEQ ID NO:1

In another embodiment, the invention provides an isolated polynucleotide comprising a sequence as set forth in SEQ ID NO:1, which is the 5′ flanking region of a CYP2C19 gene.

In another embodiment, the invention provides a kit comprising a first pair of oligonucleotide primers for amplifying the polymorphic region corresponding to position 352 of SEQ ID NO:1; a second primer pair for amplifying the polymorphic region corresponding to position 1060 of SEQ ID NO:1; a first sequence determination oligonucleotide comprising a sequence selected from the group consisting of SEQ ID NO:3; SEQ ID NO:6; SEQ ID NO:22; SEQ ID NO:23; SEQ ID NO:27; SEQ ID NO:30; SEQ ID NO:33; and SEQ ID NO:36; and a second sequence determination oligonucleotide comprising a sequence selected from the group consisting of SEQ ID NO:4; SEQ ID NO:7; SEQ ID NO:24; SEQ ID NO:25; SEQ ID NO:28; SEQ ID NO:31; SEQ ID NO:34; and SEQ ID NO:37.

### BRIEF DESCRIPTION OF THE DRAWINGS

FIG. 1 shows the sequence of the 5′ flanking region of the CYP2C19 gene as set forth in SEQ ID NO:2, with polymorphic sites underlined and highlighted in bold.

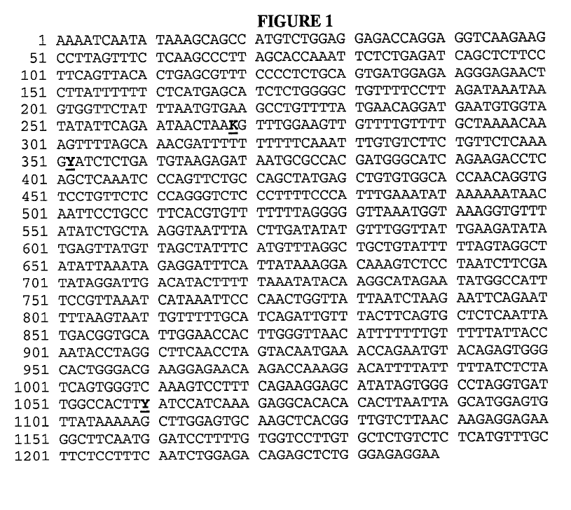

FIG. 2 shows an outline of the One Base Sequencing (OBS) principle.

### DETAILED DESCRIPTION OF THE INVENTION

The U.S. patents and publications referenced herein are hereby incorporated by reference.

For the purposes of the invention, certain terms are defined as follows.

“Gene” is defined as the genomic sequence of the CYP2C19 gene.

“Oligonucleotide” means a nucleic acid molecule preferably comprising from about 8 to about 50 covalently linked nucleotides. More preferably, an oligonucleotide of the invention comprises from about 8 to about 35 nucleotides. Most preferably, an oligonucleotide of the invention comprises from about 10 to about 25 nucleotides. In accordance with the invention, the nucleotides within an oligonucleotide may be analogs or derivatives of naturally occurring nucleotides, so long as oligonucleotides containing such analogs or derivatives retain the ability to hybridize specifically within the polymorphic region containing the targeted polymorphism. Analogs and derivatives of naturally occurring oligonucleotides within the scope of the present invention are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and the like. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372, and the like. The term “oligonucleotides” as defined herein also includes compounds which comprise the specific oligonucleotides disclosed herein, covalently linked to a second moiety. The second moiety may be an additional nucleotide sequence, for example, a tail sequence such as a polyadenosine tail or an adaptor sequence, for example, the phage M13 universal tail sequence, and the like. Alternatively, the second moiety may be a non-nucleotidic moiety, for example, a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the specific oligonucleotide, so long as the oligonucleotide retains its ability to hybridize to the polymorphic regions described herein.

An isolated polynucleotide as defined herein is a nucleic acid molecule which has been removed from its native state or synthetically manufactured. An isolated polynucleotide of the invention preferably comprises from about 50 to about 5000 covalently linked nucleotides. More preferably, an oligonucleotide of the invention comprises from about 100 to about 2000 nucleotides. Most preferably, an oligonucleotide of the invention comprises from about 200 to about 1500 nucleotides.

A polymorphic region as defined herein is a portion of a genetic locus that is characterized by at least one polymorphic site. A genetic locus is a location on a chromosome which is associated with a gene, a physical feature, or a phenotypic trait. A polymorphic site is a position within a genetic locus at which at least two alternative sequences have been observed in a population. A polymorphic region as defined herein is said to “correspond to” a polymorphic site, that is, the region may be adjacent to the polymorphic site on the 5′ side of the site or on the 3′ side of the site, or alternatively may contain the polymorphic site. A polymorphic region includes both the sense and antisense strands of the nucleic acid comprising the polymorphic site, and may have a length of from about 100 to about 5000 base pairs. For example, a polymorphic region may be all or a portion of a regulatory region such as a promoter, 5′ UTR, 3′ UTR, an intron, an exon, or the like. A polymorphic or allelic variant is a genomic DNA, cDNA, mRNA or polypeptide having a nucleotide or amino acid sequence that comprises a polymorphism. A polymorphism is a sequence variation observed at a polymorphic site, including nucleotide substitutions (single nucleotide polymorphisms or SNPs), insertions, deletions, and microsatellites. Polymorphisms may or may not result in detectable differences in gene expression, protein structure, or protein function. Preferably, a polymorphic region of the present invention has a length of about 1000 base pairs. More preferably, a polymorphic region of the invention has a length of about 500 base pairs. Most preferably, a polymorphic region of the invention has a length of about 200 base pairs.

A haplotype as defined herein is a representation of the combination of polymorphic variants in a defined region within a genetic locus on one of the chromosomes in a chromosome pair. A genotype as used herein is a representation of the polymorphic variants present at a polymorphic site.

Methods of predicting an individual human's capacity to metabolize drugs which are substrates for the CYP2C19 enzyme are encompassed by the present invention. In the methods of the invention, the presence or absence of at least three polymorphic variants of the nucleic acid of SEQ ID NO:1 are detected to determine the individual's haplotype for those variants. Specifically, in a first step, a nucleic acid is isolated from biological sample obtained from the human. Any nucleic-acid containing biological sample from the human is an appropriate source of nucleic acid for use in the methods of the invention. For example, nucleic acid can be isolated from blood, saliva, sputum, urine, cell scrapings, biopsy tissue, and the like. In a second step, the nucleic acid is assayed for the presence or absence of at least three allelic variants of the polymorphic regions of the nucleic acid of SEQ ID NO:1 described above. Specifically, a haplotype is constructed for at least two polymorphic sites in the 5′ regulatory region of the CYP2C19 gene in the method of the invention. The polymorphic sites may be selected from the group consisting of positions 269, 352, and 1060 of SEQ ID NO:1. Preferably, at least two polymorphic sites on each chromosome in the chromosome pair of the human are assayed in the method of the invention, so that the zygosity of the individual for the particular polymorphic variant may be determined.

Any method may be used to assay the nucleic acid, that is, to determine the sequence of the polymorphic region, in this step of the invention. For example, any of the primer extension-based methods, ligase-based sequence determination methods, mismatch-based sequence determination methods, sequencing methods, or microarray-based sequence determination methods described above may be used, in accordance with the present invention. Alternatively, such methods as restriction fragment length polymorphism (RFLP) detection, single strand conformation polymorphism detection (SSCP), PCR-based assays such as the Taqman® PCR System (Applied Biosystems) may be used.

The oligonucleotides of the invention may be used to determine the sequence of the polymorphic regions of SEQ ID NO:1. In particular, the oligonucleotides of the invention may comprise sequences as set forth in SEQ ID NO:2; SEQ ID NO:3; SEQ ID NO:4; SEQ ID NO:5; SEQ ID NO:6; SEQ ID NO:7; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; and SEQ ID NO:37.

Those of ordinary skill will recognize that oligonucleotides complementary to the polymorphic regions described herein must be capable of hybridizing to the polymorphic regions under conditions of stringency such as those employed in primer extension-based sequence determination methods, restriction site analysis, nucleic acid amplification methods, ligase-based sequencing methods, methods based on enzymatic detection of mismatches, microarray-based sequence determination methods, and the like. The oligonucleotides of the invention may be synthesized using known methods and machines, such as the ABI™3900 High Throughput DNA Synthesizer and the Expedite™ 8909 Nucleic Acid Synthesizer, both of which are available from Applied Biosystems (Foster City, Calif.).

The oligonucleotides of the invention may be used, without limitation, as in situ hybridization probes or as components of diagnostic assays. Numerous oligonucleotide-based diagnostic assays are known. For example, primer extension-based nucleic acid sequence detection methods are disclosed in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; WO 01/20039; and the like. Primer extension-based nucleic acid sequence detection methods using mass spectrometry are described in U.S. Pat. Nos. 5,547,835; 5,605,798; 5,691,141; 5,849,542; 5,869,242; 5,928,906; 6,043,031; 6,194,144, and the like. The oligonucleotides of the invention are also suitable for use in ligase-based sequence determination methods such as those disclosed in U.S. Pat. Nos. 5,679,524 and 5,952,174, WO 01/27326, and the like. The oligonucleotides of the invention may be used as probes in sequence determination methods based on mismatches, such as the methods described in U.S. Pat. Nos. 5,851,770; 5,958,692; 6,110,684; 6,183,958; and the like. In addition, the oligonucleotides of the invention may be used in hybridization-based diagnostic assays such as those described in U.S. Pat. Nos. 5,891,625; 6,013,499; and the like.

The oligonucleotides of the invention may also be used as components of a diagnostic microarray. Methods of making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; WO 01/29259; and the like.

Each of the PCR primer pairs of the invention may be used in any PCR method. For example, a PCR primer pair of the invention may be used in the methods disclosed in U.S. Pat. Nos. 4,683,195; 4,683,202, 4,965,188; 5,656,493; 5,998,143; 6,140,054; WO 01/27327; WO 01/27329; and the like. The PCR pairs of the invention may also be used in any of the commercially available machines that perform PCR, such as any of the GeneAmp® Systems available from Applied Biosystems.

The isolated polynucleotide of the invention comprises the sequence as set forth in SEQ ID NO:1. The isolated polynucleotide of the invention may be used as a standard or control in methods and kits that detect or identify polymorphisms in the CYP2C19 gene. In particular, the isolated polynucleotide of the invention may be used in the methods and kits described herein. Alternatively, the isolated polynucleotide of the invention may be used as a component of an expression vector which also comprises a nucleic acid encoding a cytochrome P450 enzyme, preferably the coding sequence of CYP2C19, to assay whether a test compound is a substrate for the enzyme. In this way the test compound's ability to interact with the 5′ flanking region of the CYP2C19 gene may be determined in vitro. Methods of constructing such expression vectors and assays are well known in the art.

The invention is also embodied in a kit comprising at least one oligonucleotide primer pair of the invention. Preferably, the kit of the invention comprises at least two oligonucleotide primer pairs, wherein each primer pair is complementary to a different polymorphic region of the nucleic acid of SEQ ID NO:1. More preferably, the kit of the invention comprises at least three oligonucleotide primer pairs suitable for amplification of polymorphic regions corresponding to positions 269, 352, and 1060 of SEQ ID NO:1. This embodiment may optionally further comprise a sequence determination oligonucleotide for detecting a polymorphic variant at any or all of the polymorphic sites corresponding to positions 269, 352, and 1060 in SEQ ID NO:1. The kit of the invention may also comprise a polymerizing agent, for example, a thermostable nucleic acid polymerase such as those disclosed in U.S. Pat. Nos. 4,889,818; 6,077,664, and the like. The kit of the invention may also comprise chain elongating nucleotides, such as dATP, dTTP, dGTP, dCTP, and dITP, including analogs of DATP, dTTP, dGTP, dCTP and dITP, so long as such analogs are substrates for a thermostable nucleic acid polymerase and can be incorporated into a growing nucleic acid chain. The kit of the invention may also include chain terminating nucleotides such as ddATP, ddTTP, ddGTP, ddCTP, and the like. In a preferred embodiment, the kit of the invention comprises at least two oligonucleotide primer pairs, a polymerizing agent, chain elongating nucleotides, at least two sequence determination oligonucleotides and at least one chain terminating nucleotide. The kit of the invention may optionally include buffers, vials, microtiter plates, and instructions for use.

In one specific embodiment, the invention provides a kit comprising a pair of oligonucleotide primers suitable for amplifying the polymorphic region corresponding to position 352 of the CYP2C19 gene 5′ flanking region as set forth in SEQ ID NO:1, a primer pair suitable for amplifying the polymorphic region corresponding to position 1060 of the CYP2C19 gene 5′ flanking region as set forth in SEQ ID NO:1; a sequence determination oligonucleotide comprising a sequence selected from the group consisting of SEQ ID NO:3; SEQ ID NO:6; SEQ ID NO:22; SEQ ID NO:23; SEQ ID NO:27; SEQ ID NO:30; SEQ ID NO:33; and SEQ ID NO:36; and a sequence determination oligonucleotide comprising a sequence selected from the group consisting of SEQ ID NO:4; SEQ ID NO:7; SEQ ID NO:24; SEQ ID NO:25; SEQ ID NO:28; SEQ ID NO:31; SEQ ID NO:34; and SEQ ID NO:37. The primer pairs of this embodiment are preferably selected from the group consisting of SEQ ID NO:8 and SEQ ID NO:9, SEQ ID NO:16 and SEQ ID NO:17, and SEQ ID NO:18 and SEQ ID NO:19 (for amplification of the polymorphic region corresponding to position 352 of SEQ ID NO:1); SEQ ID NO:10 and SEQ ID NO:11; SEQ ID NO:12 and SEQ ID NO:13; and SEQ ID NO:14 and SEQ ID NO:15 (for amplification of the polymorphic region corresponding to position 1060 of SEQ ID NO:1).

When the kit comprises the oligonucleotide primer pairs set forth in SEQ ID NO:8 and SEQ ID NO:9 or SEQ ID NO:16 and SEQ ID NO:17, the kit of the invention may further optionally comprise a sequence determination oligonucleotide for detection of the polymorphic region corresponding to position 269 of SEQ ID NO:1, said sequence determination oligonucleotide being selected from the group consisting of SEQ ID NO:2; SEQ ID NO:5; SEQ ID NO:20; SEQ ID NO:21; SEQ ID NO:26; SEQ ID NO:29; SEQ ID NO:32; and SEQ ID NO:35.

The examples set forth below are provided as illustration and are not intended to limit the scope and spirit of the invention as specifically embodied therein.

### EXAMPLE 1

### Phenotypes of Study Participants

The study was performed in accordance with the principles stated in the Declaration of Helsinki as reviewed in Tokyo 1975 and Venice 1983, Hong Kong 1989 and Somerset West 1996. Subjects were preferably not related to each other. Based on questioning, individuals having one of the following were excluded: a medical condition judged to influence liver function or requiring pharmacological treatment; any on-going disease; intake of any drug, except oral contraceptives, during one week prior to the study; breast-feeding or pregnancy. No physical examination was performed. For these experiments, a single oral dose of 20 mg omeprazole (Losec, AstraZeneca) was given in the morning after an overnight fast. The bladder was emptied before drug intake. A single blood sample was collected 3 hours after drug intake.

In the first part of the study, approximately 90 samples (Swedish Caucasians) were selected as set forth in Table 1, on the basis of the following assumptions: if the distribution of an unknown polymorphism will be 25% for a homozygote, a sample size of approximately 40 “UEM” will be able to detect an increase in this specific genotype (homozygote) by 28% (α=5% (two-tailed), power=80%). If it is assumed that the distribution of an unknown polymorphism will be 10% for a homozygote, a sample size of approximately 40 “UEM” will be able to detect an increase in this specific genotype (homozygote) by 21% (α-5% (two-tailed), power=80%). The samples were selected with regard to their phenotyped metabolic ratios (MR) of omeprazole. Available genotype information for all samples was provided.

Individuals with known defective alleles, i.e. CYP2C19*2 and CYP2C19*3 were excluded. However, a few extra samples genotyped for any of the alleles mentioned above were included as outlier controls.

TABLE 1

| # of samples | MR | Phenotype |
|---|---|---|
| 9 | <0.2 | UEM |
| 48 | 0.2-0.8 | fast EM |
| 25 | 0.81-12.6 | slow EM |
| 0 | >12.6 | PM |

The first part of the study resulted in identification of three SNPs in the 5′ flanking region of the CYP2C19 gene. Oligonucleotides containing these SNPs are shown in Table 2.

TABLE 2

| Polymorphic | | Nucleotide |
|---|---|---|
| Site | Sequence | Change |
| 269 | SEQ ID NO:2: ACTAATGTTTG | T variant |
| | SEQ ID NO:5: ACTAAGGTTTG | G variant |
| 352 | SEQ ID NO:3: CAAAGCATCTC | C variant |
| | SEQ ID NO:6: CAAAGTATCTC | T variant |
| 1060 | SEQ ID NO:4: CACTTTATCCA | T variant |
| | SEQ ID NO:7: CACTTCATCCA | C variant |

In the second part of the study, 71 samples with a more normal phenotypic distribution were used. Also, no exclusion of individuals with known defective alleles or duplications was done.

TABLE 3

| # of samples | MR | Phenotype |
|---|---|---|
| 2 | <0.2 | UEM |
| 45 | 0.2-0.8 | fast EM |
| 19 | 0.81-12.6 | slow EM |
| 5 | >12.6 | PM |

### EXAMPLE 2

### CYP2C19 Genetic Analysis

White blood cells isolated from a blood sample drawn from the brachial vein serve as the source of the genomic DNA for the analyses. The DNA is extracted by guanidine thiocyanate method or QlAamp Blood Kit (QIAGEN, Venlo, The Netherlands). The genes included in the study were amplified by PCR and the DNA sequences were determined by the technology most suitable for the specific fragment. All genetic analyses were performed according to Good Laboratory Practice and Standard Operating Procedures. Case Report Forms were designed and used for clinical and genetic data collection. Data was entered and stored in a relational database at Gemini Genomics AB, Uppsala. To secure consistency between the Case Report Forms and the database, data was checked either by double data entry or proofreading. After a Clean File was declared the database was protected against changes. By using the program Stat/Transfer™ the database was transferred to SAS data sets. The SAS™ system was used for tabulations and statistical evaluations. Genotypes were also correlated against the metabolic ratio.

PCR-fragments were amplified with TaqGOLD polymerase (Applied Biosystems) using Robocycler (Stratagene) or GeneAmp PCR system 9700 (Applied Biosystems). Preferentially, the amplified fragments were 300-400 bp, and the region to be read did not exceed 300 bp for full sequencing and did not exceed 60 bp for One Base Sequencing (OBS). PCR reactions were carried out according to the basic protocol set forth in Table 4, with modifications as indicated in Table 5 for specific primer pairs, which are shown in Table 6. For the GeneAmp PCR 9700 machine the profile used was 10 minutes at 95°, 40×(45 seconds at 90°, 45 seconds at 60°, 45 seconds at 72°), 5 minutes at 72° and 22° until removed.

TABLE 4

| | Stock | |
|---|---|---|
| Solution | Concentration | PCR (μl) |
| H₂O | | 33.2 |
| PCR buffer | 10× | 5.0 |
| MgCl₂ | 25 mM | 2.0 |
| dNTP | 2.5 mM | 2.5 |
| primer 1 | 10 μM | 1.0 |
| primer 2 | 10 μM | 1.0 |
| Taq-gold | 5 μ/μl | 0.3 |
| polymerase | | |
| DNA samples | 2 ng/μl | 5.0 |
| TOTAL | | 50.0 |

TABLE 5

| SEQ ID | Polymorphic | | |
|---|---|---|---|
| NO:s | Site | Modification from basic protocol (Table 4) | Detection method |
| 8, 9 | 269 & 352 | 3 μl MgCl₂, 62° annealing temperature | Full sequencing |
| 16, 17 | 269 & 352 | 4 μl MgCl₂, 52° annealing temperature, 50 | Full sequencing & OBS |
| | | cycles | |
| 18, 19 | 352 | 4 μl MgCl₂, 55° annealing temperature, 50 | Full sequencing & OBS |
| | | cycles | |
| 10, 11 | 1060 | 3 μl MgCl₂, 62° annealing temperature | Full sequencing & OBS |
| 12, 13 | 1060 | 3 μl MgCl₂, 58° annealing temperature | Full sequencing |
| 14, 15 | 1060 | 3 μl MgCl₂, 58° annealing temperature | Full sequencing |

TABLE 6

| Polymorphic | | |
|---|---|---|
| Site | Primer Pair | |
| 269 & 352 | SEQ ID NO:8 | CAGGAGGTCAAGAAGCCTTAGT |
| | SEQ ID NO:9 | CCATCGTGGCGCATTATCT |
| 1060 | SEQ ID NO:10 | ACGGTGCATTGGAACCACTT |
| | SEQ ID NO:11 | CCCAGAGCTCTGTCTCCAGAT |
| 1060 | SEQ ID NO:12 | AGTGGGCACTGGGACGA |
| | SEQ ID NO:13 | GATCCATTGAAGCCTTCTCC |
| 1060 | SEQ ID NO:14 | GTAATTGTTTTTGCATCAGATTG |
| | SEQ ID NO:15 | TCCATGCTAATTAAGTGTGTGTG |
| 269 & 352 | SEQ ID NO:16 | CTGAGATCAGCTCTTCCTTCAG |
| | SEQ ID NO:17 | AGGCAGGAATTGTTATTTTTTATA |
| 352 | SEQ ID NO:18 | TGGGGCTGTTTTCCTAGAT |
| | SEQ ID NO:19 | ATTTAACCCCCTAAAAAAACAC |

The optimized condition specified in Table 4 were required to distinguish CYP2C19 from the closely related gene-family members CYP2C8, CYP2C9 and CYP2C18. Use of the basic protocol will lead to problems when amplifying CYP2C19-specific amplicons of 300-400 bp containing the polymorphisms of interest, unless a nested PCR approach is carried out. The nested PCR approach was not used because of the high risk of contamination when using a nested PCR approach and the high risk of typing errors as a consequence. The modifications shown in Table 5 were optimized and reaction parameters were balanced in such a way that nested PCR was avoided.

For full sequencing, one of the PCR-primers in a primer pair was designed for sequencing by addition of a 29 nucleotide tail complementary to M13 at its 5′-end, namely the nucleotides AGTCACGACGTTGTAAAACGACGGCCAGT. Thus, the entire PCR-product was sequenced from the tailed PCR-primer.

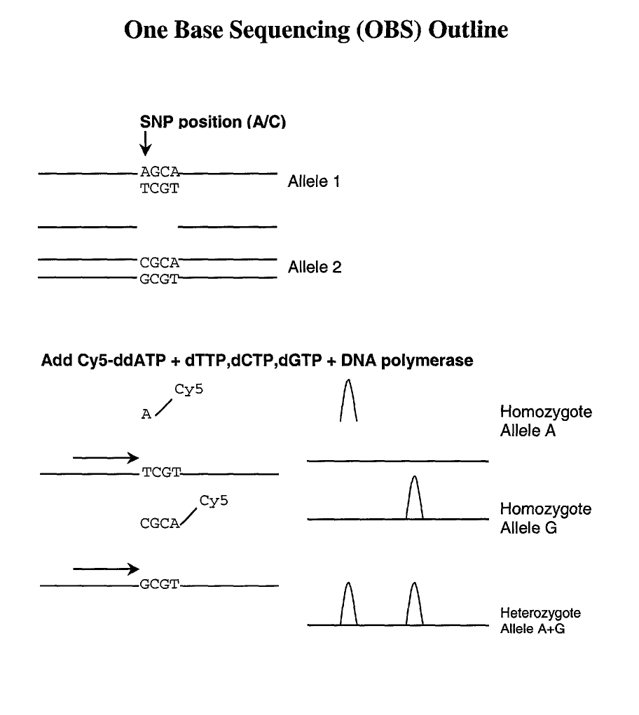

The OBS method as used herein is described in commonly assigned international patent application number PCT/GB01/00828. Briefly, the OBS method is a mini sequencing/primer extension variant, which uses a unique mixture of three dNTPs and one ddNTP. A sequencing primer is positioned adjacent or close to a polymorphic position, e.g., a SNP. The extension from the sequencing primer annealed to a single stranded PCR product continues until a ddNTP is incorporated. For example, when detecting an A/C SNP using a ddATP terminator, the extension will stop at the SNP if an A is present but will continue to the next A in the sequence if a C is present. Thus, a heterozygote sample will produce two extension products of different defined lengths (see FIG. 2).

The additional oligonucleotides set forth in Tables 7 through 9 were identified as being suitable for detection of the SNPs at positions 269, 352, and/or 1060 of the 5′ flanking region of the CYP2C19 gene as depicted in SEQ ID NO:1.

Table 7 sets forth oligonucleotides representing the non-coding (anti-sense) strand complementary to the polymorphic region corresponding to the polymorphisms found in the study population. The underlined letter indicates polymorphic position in the sequence context. Numbers inside brackets are calculated from the transcriptional start. All sequences are shown in 5′ to 3′ direction.

TABLE 7

| Polymorphic | | |
|---|---|---|
| Site | Sequence | Note |
| 269 | SEQ ID NO:20: CAAACATTAGT | Antisense |
| | | A variant |
| | SEQ ID NO:21: CAAACCTTAGT | Antisense |
| | | C variant |
| 352 | SEQ ID NO:22: GAGATGCTTTG | Antisense |
| | | G variant |
| | SEQ ID NO:23: GAGATACTTTG | Antisense |
| | | A variant |
| 1060 | SEQ ID NO:24: TGGATAAAGTG | Antisense |
| | | A variant |
| | SEQ ID NO:25: TGGATGAAGTG | Antisense |
| | | G variant |

The sequences of Table 8 represent the 5′-sequence to the polymorphic sites on the coding (sense) strand (SEQ ID NO:s 26-28) and non-coding (anti-sense) strand (SEQ ID NO:s 29-31). Numbers inside brackets are calculated from the transcriptional start. All sequences are shown in 5′ to 3′ direction.

TABLE 8

| Polymor- | | |
|---|---|---|
| phic Site | Sequence | Note |
| 269 | SEQ ID NO:26: TCAGAATAACT | Sense 5′ |
| | SEQ ID NO:29: AGTTATTCTGA | Antisense 5′ |
| 352 | SEQ ID NO:27: TCTGTTCTCAA | Sense 5′ |
| | SEQ ID NO:30: TTGAGAACAGA | AntiSense 5′ |
| 1060 | SEQ ID NO:28: TGATTGGCCAC | Sense 5′ |
| | SEQ ID NO:31: GTGGCCAATCA | Antisense 5′ |

The sequences of Table 9 represent the 3′-sequence to the polymorphic sites on the non-coding (anti-sense) strand (SEQ ID NO:s 32-34) and the coding (sense) strand (SEQ ID NO:s 35-37). Numbers inside brackets are calculated from the transcriptional start. All sequences are shown in 5′ to 3′ direction.

TABLE 9

| Polymor- | | |
|---|---|---|
| phic Site | Sequence | Note |
| 269 | SEQ ID NO:32: ACTTCCAAAC | Antisense 3′ |
| | SEQ ID NO:35: GTTTGGAAGT | Sense 3′ |
| 352 | SEQ ID NO:33: ACATCAGAGAT | Antisense 3′ |
| | SEQ ID NO:36: ATCTCTGATGT | Sense 3′ |
| 1060 | SEQ ID NO:34: CTTTGATGGAT | Antisense 3′ |
| | SEQ ID NO:37: ATCCATCAAAG | Sense 3′ |

### EXAMPLE 3

### Haplotype and Genotype Analyses

Haplotype analysis could be performed on a total of 232 individuals. This analysis was performed using software based on maximum likelihood methodology and using the EM algorithm of Excoffier et al. (1995), Mol Biol Evol. 12:921-927. In total 5 likely haplotypes were identified by the program. One of these occurred only six times in the study population and has been excluded from the study due to its low frequency. The characterization of each haplotype is presented in Table 10, and the frequency of each haplotype is set forth in Table 11. From the haplotype information two different kinds of variables were created: one variable was formed as a haplotype combination variable (HTYPE). This variable has the value H1/H2 when the subject has haplotypes 1 and 2, etc. Variables H1, H2, H3 and H4 are haplotype annotations that denote the number of copies of that particular haplotype for the subject, e.g., for a subject with haplotype H1/H2 the variables H1, H2, H3 and H4 will be 1, 1, 0 and 0, respectively. Each of these variables can thus take on the values 0, 1 or 2. Only the four most frequent haplotypes were considered when those variables were formed.

TABLE 10

| | Nucleotide at polymorphic | | |
|---|---|---|---|
| | position: | | |
| Haplotype | 269 | 352 | 1060 |
| CYP2C19 5′ | T | C | T |
| flanking (SEQ ID | | | |
| NO:1) | | | |
| H1 | T | C | T |
| (TCT) | | | |
| H2 | T | T | T |
| (TTT) | | | |
| H3 | T | C | C |
| (TCC) | | | |
| H4 | G | C | T |
| (GCT) | | | |

TABLE 11

| | Haplotype | | | | |
|---|---|---|---|---|---|
| Haplotype | frequency | P-value (Sp) | Note | | |
| H1 | 60% | 0.0076 | H1/H1 | n = 60 | mr50 = 0.485 |
| | | | H1/— | n = 63 | mr50 = 0.63 |
| | | | —/— | n = 30 | mr50 = 0.97 |
| H2 | 17% | 0.0004 | H2/H2 | n = 4 | mr50 = 0.25 |
| | | | H2/— | n = 44 | mr50 = 0.485 |
| | | | —/— | n = 105 | mr50 = 0.64 |
| H3 | 17% | <0.0001 | H3/H3 | n = 7 | mr50 = 16.86 |
| | | | H3/— | n = 39 | mr50 = 0.88 |
| | | | —/— | n = 107 | mr50 = 0.47 |
| H4 | 6% | 0.3947 | H4/H4 | n = 2 | mr50 = 1.5 |
| | | | H4/— | n = 14 | mr50 = 0.755 |
| | | | —/— | n = 137 | mr50 = 0.56 |

Table 11 also sets forth the statistical p-values (Spearman correlation) between CYP2C19 haplotypes H1-H4 and mr(omeprazole), where mr50 is an abbreviation for metabolic ratio of the 50^{th} percentile.

Table 12 sets forth a summary of the predictive haplotypes found in the study described in Examples 1 and 2.

TABLE 12

| | | Metabolic | |
|---|---|---|---|
| Haplotype | Frequency | capacity | Note |
| H1 | 66% | EM | H1 & H4 |
| H2 | 17% | UEM/EM | |
| H3 | 17% | PM | In 98% LD with CYP2C19*2 |
| | | | (52 samples/53 samples) |

Table 13 shows CYP2C19 genotype markers for haplotype combinations and their predicted metabolic ratios based on 144 samples.

TABLE 13

| CYP2C19 genotype | | | | | % of haplotypes | MR-range |
|---|---|---|---|---|---|---|
| 5D6:352 | 2D6:1060 | HTYPE | Marker for | MR (Ome) | in MR-range | (min-max) |
| T | T | H2/H2 | UEM/EM | <0.4 | 100% (4/4) | 0.15-0.33 |
| C/T | T | H1/H2 | UEM & EM | <0.8 | 90% (28/31) | 0.12-2.62 |
| C | T | H1/H1 | EM | 0.2-0.8 | 79% (50/63) | 0.17-2.90 |
| C/T | C/T | H2/H3 | EM & (IM) | 0.4-2.0 | 92% (11/12) | 0.36-1.75 |
| C | C/T | H1/H3 | EM &IM | 0.4-7.0 | 93% (25/27) | 0.03-11.87 |
| C | C | H3/H3 | PM | >7.0 | 86% (6/7) | 1.28-23.75 |

While the invention has been described in terms of the specific embodiments set forth above, those of skill will recognize that the essential features of the invention may be varied without undue experimentation and that such variations are within the scope of the appended claims.

1. A method for determining a human's capacity to metabolize a substrate of a CYP2C19 enzyme, said method comprising the steps of:
a) isolating single stranded nucleic acids from the human, said nucleic acids encoding 5′ flanking regions of CYP2C19 genes present on each homologous chromosome 10 of the human, wherein said region is represented by a sequence as set forth in SEQ ID NO:1; and
b) detecting at least two polymorphisms within the region, wherein the polymorphisms are nucleotides present at polymorphic sites represented by positions 352 and 1060 of SEQ ID NO:1.

2. A sequence determination oligonucleotide suitable for detecting a polymorphic site in a 5′ flanking region of a CYP2C19 gene, said oligonucleotide comprising a sequence selected from the group consisting of SEQ ID NO:2; SEQ ID NO:3; SEQ ID NO:4; SEQ ID NO:5; SEQ ID NO:6; SEQ ID NO:7; SEQ ID NO:20; SEQ ID NO:21, SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33, SEQ ID NO:34; SEQ ID NO:35; SEQ ID NO:36; and SEQ ID NO:37.

3. An oligonucleotide primer pair suitable for amplifying a 5′ flanking region of a CYP2C19 gene, said primer pair having sequences selected from the group consisting of: SEQ ID NO:8 and SEQ ID NO:9; SEQ ID NO:10 and SEQ ID NO:11; SEQ ID NO:12 and SEQ ID NO:13; SEQ ID NO:14 and SEQ ID NO:15; SEQ ID NO:16 and SEQ ID NO:17; and SEQ ID NO:18 and SEQ ID NO:19.

4. An isolated polynucleotide comprising a sequence as set forth in SEQ ID NO:1.

5. A kit comprising:
a) a first pair of oligonucleotide primers for amplifying the polymorphic region corresponding to position 352 of SEQ ID NO:1;
b) a second primer pair for amplifying the polymorphic region corresponding to position 1060 of SEQ ID NO:1;
c) a first sequence determination oligonucleotide comprising a sequence selected from the group consisting of SEQ ID NO:3; SEQ ID NO:6; SEQ ID NO:22; SEQ ID NO:23; SEQ ID NO:27; SEQ ID NO:30; SEQ ID NO:33; and SEQ ID NO:36; and
d) a second sequence determination oligonucleotide comprising a sequence selected from the group consisting of SEQ ID NO:4; SEQ ID NO:7; SEQ ID NO:24; SEQ ID NO:25; SEQ ID NO:28; SEQ ID NO:31; SEQ ID NO:34; and SEQ ID NO:37.

6. The kit of claim 5, wherein the first primer pair selected from the group consisting of SEQ ID NO:8 and SEQ ID NO:9; SEQ ID NO:16 and SEQ ID NO:17; and SEQ ID NO:18 and SEQ ID NO:19; and the second primer pair is selected from the group consisting of SEQ ID NO:10 and SEQ ID NO:11; SEQ ID NO:12 and SEQ ID NO:13; and SEQ ID NO:14 and SEQ ID NO:15.
